(19) United States
(12) Patent Application Publication
(10) Pub. No.: US 2011/0275598 A1
(43) Pub. Date: Nov. 10, 2011

(54) COSMETIC USE OF PROANTHOCYANIDIN A2

(75) Inventors: Carlo Felice Fornada, Milano (IT); Alessandro Mazzetti, Milano (IT); Stefano Togni, Milano (IT)

(73) Assignee: DERMOGYN SRL, Milano (IT)

(21) Appl. No.: 13/142,661

(22) PCT Filed: Jan. 27, 2010
(86) PCT No.: PCT/EP2010/050907

§ 371 Date: Jun. 29, 2011

(30) Foreign Application Priority Data
Feb. 2, 2009 (IT) MI2009A000122

(51) Int. Cl. A61K 8/55; A61Q 19/08; A61K 8/49

(52) U.S. Cl. 514/77; 514/453

(57) ABSTRACT

The use of proanthocyanidin A2 and of its complexes with phospholipids for the reshaping treatment of the breast and the anti-age treatment of the neck and the décolleté is described.

The present invention relates to the cosmetic use of proanthocyanidin A2 and complexes thereof, and more particularly relates to the cosmetic use of proanthocyanidin A2 and complexes thereof for the re-shaping treatment of the breast and the anti-age treatment of the neck and the décolleté.

Proanthocyanidin A2 (herein after also referred to as PA2) is a flavonoid of natural origin isolated from the bark of Aesculus hippocastanus.

PA2, chemical name [2R-(2α,3α,8β,14β,15R)]-2,8-bis(3,4-dihydroxy-phenyl)-3,4-dihydro-8,14-methan-2H,14H-1-benzopyran[7,8-d][1,3]benzodioxocin-3,5,11,13,15-pentol dihydrate, is used as such or in the form of complex with phospholipids for its pharmacological and cosmetic properties.

Pharmaceutical and/or cosmetic compositions comprising PA2 are described in EP 0 210 785 (Inverni della Beffa SpA), EP 0 694 305 (Indena SpA), EP 1 086 693 (Kyowa Hakko Kogyo Co. Ltd.), and US 2004/0234480 (Cognis Corporation).

WO 02/102349 (LG Household & Health Care Ltd.) describes the use of procyanidin oligomers, consisting of 3-12 units with an average molecular weight of 1,000-2,000 and an average polymerization degree of 4.5-6.0, for the prevention and treatment of skin wrinkles.

PA2 complexes with phospholipids and their pharmaceutical use for atherosclerosis treatment are described in EP 1 140 115 (Indena SpA).

We have now found that PA2 and its complexes with phospholipids are effective for re-shaping the breast and as anti-age agent for the neck and the décolleté.

Therefore, object of the present invention is the cosmetic use of proanthocyanidin A2 and its complexes with phospholipids for the re-shaping treatment of the breast and the anti-age treatment of the neck and the décolleté.

Cosmetic compositions comprising PA2 or PA2 complexed with phospholipids for the re-shaping treatment of the breast and the anti-age treatment of the neck and the décolleté are a further object of the present invention.

PA2 complexed with phospholipids, and still more preferably PA2 complexed with phosphatidylcholine, is preferred for the use and for the cosmetic compositions object of the present invention.

The cosmetic compositions object of the present invention are conventional topic compositions such as cream, emulsions, lotions etc. comprising an amount of PA2 or complex thereof ranging from 0.1% to 5% w/w, preferably from 0.5% to 3% w/w, still more preferably from 2% to 2.5% w/w.

The cosmetic compositions can also contain usual excipients for topical cosmetic use such as surface-active agents, emulsifying agent, waxes, anti-oxidants, stabilizers, perfumes, preservatives, polymers, solvents, etc. and they are prepared according to conventional techniques.

The re-shaping effect on the breast of the cosmetic compositions object of the present invention has the anti-age effect on the neck and the décolleté as its perfect complement. However the compositions object of the present invention can be used for the re-shaping treatment of the breast only as well as for the anti-age treatment of the neck and the décolleté only. Cosmetic compositions comprising PA2 or PA2 complexed with phospholipids for the re-shaping treatment of the breast and cosmetic compositions comprising PA2 or PA2 complexed with phospholipids for the anti-age treatment of the neck and the décolleté are therefore further objects of the present invention.

It is worth underlining that the cosmetic effect on the breast, neck and decolleté can be achieved with the use of PA2 or its complexes with phospholipids as the only cosmetic active ingredient. For the re-shaping and anti-age effect according to the use object of the present invention, it is not necessary to make use of associations with other cosmetic agents or of extracts enriched with other products.

The cosmetic compositions object of the present invention are applied on the breast and/or on the neck and décolleté from 1 to 4 times a day.

Optimal results can be obtained by applying the cosmetic compositions twice a day for at least 4 weeks.

Examples of cosmetic formulations according to the present invention are reported in the following table (amounts are expressed as percentage by weight):

| | A | B | C |
|---|---|---|---|
| Water | 75.600 | 71.980 | 61.524 |
| Acrylamide/sodium | 0.750 | | |
| acryloyldimethyltaurate | | | |
| copolymer | | | |
| Cetearyl alcohol | | 2.600 | 3.400 |
| Algae | | | 0.075 |
| Aminomethyl propanol | 0.100 | 0.120 | 0.150 |
| Butylmethoxydibenzoylmethane | | | 0.500 |
| Butylparaben | 0.035 | 0.035 | 0.0365 |
| Carbomer | | 0.150 | 0.250 |
| Lysine carboxymethyl cisteinate | 0.750 | 0.750 | 0.750 |
| Cyclopentansiloxane | 2.880 | 4.800 | 2.880 |
| Coco-caprylate/caprate | | 5.000 | |
| Acrylates/C10-30 alkyl | 0.200 | | |
| acrylate crosspolymer | | | |
| Dimethicone/vinyl | 0.120 | 0.200 | 0.120 |
| dimethicone crosspolimer | | | |
| Dicaprylyl ether | 7.500 | | 7.000 |
| Disodium EDTA | 0.100 | 0.100 | 0.100 |
| Rosmarinus officinalis extract | 0.001 | 0.001 | 0.0005 |
| Ethylhexylmethoxycinnamate | | | 2.000 |
| Ethylparaben | 0.025 | 0.025 | 0.0265 |
| Ethoxydiglycol | 2.000 | 2.000 | 2.000 |
| Phenoxyethanol | 0.350 | 0.350 | 0.3515 |
| Glyceryl stearate | | 2.600 | 3.400 |
| Glycerin | 2.500 | 2.500 | 3.3675 |
| Glycine soja oil | 0.099 | 0.099 | 0.0495 |
| Imidazolidinyl urea | 0.300 | 0.300 | 0.300 |
| Isohexadecane | 0.450 | | |
| Cetearyl isononanoate | | 2.000 | |
| Methylparaben | 0.075 | 0.075 | 0.0765 |
| Olea europaea | 2.500 | | 2.500 |
| Oxidoreductase | | | 0.0150 |
| PA2 complexed with | 2.250 | 2.250 | 2.250 |
| phosphatidylcholine | | | |
| Persea gratissima | 0.500 | | 1.000 |
| Polysorbate 80 | 0.150 | | |
| Potassium sorbate | 0.250 | 0.250 | 0.250 |
| Parfum | 0.200 | 0.200 | 0.200 |
| Propylparaben | 0.015 | 0.015 | 0.01650 |
| Potassium palmitoyl | 1.300 | 1.700 | |
| hydrolyzed wheat protein | | | |
| Glycin soja protein | | | 0.0150 |
| Pullulan | | | 0.375 |
| Tocopheryl acetate | 0.300 | 0.300 | 0.300 |
| Caprylic/capric triglyceride | | | 3.000 |

The effectiveness of the compositions object of the present invention was demonstrated with a study conducted on 25 female healthy subjects, age range 35-53 years (mean=43) with mild-moderate breast slackness.

The experiment foresaw 3 visits: a basal visit (T0), a visit after 2 weeks treatment (T2) and a final visit after 4 weeks treatment (T4). Moreover a short term evaluation (only for torsiometry and hydration) was performed 30 minutes and 2 hours after the first application of the product (shock treatment).

Composition A was applied twice a day during the first and the second week of the study (shock treatment), while the maintenance treatment (composition B) was applied twice a day during the third and the fourth week.

The following evaluations were performed monolaterally:

clinical evaluations (breast firmness and cutaneous microrelief)
morphometrical evaluations (breast coordinates)
instrumental evaluations (hydration and torsiometry)

### Clinical Evaluations

At the end of the study a significant improvement (+25.6%) of the breast firmness compared with basal conditions was observed.

The cutaneous microrelief looked more regular already after 2 weeks treatment with a decrease of the irregularity corresponding to 24% compared with basal value. The decrease resulted even more significant at the end of the 4 weeks treatment (−32.5%).

### Morphometrical Evaluations

The upright position of mammary coordinates resulted significantly modified already after 2 weeks treatment with raised as well as with lowered arms.

In particular:

Raised arms: the distance between the manubrium sterni and the nipple decreased of 3.9% from basal value (mean value decreased from 17.67 cm to 16.97 cm) after two weeks treatment and of 6.8% (−1.21 cm) after 4 weeks.

Lowered arms: the distance between the manubrium sterni and the nipple decreased of 4% from basal value (mean value decreased from 21.06 cm to 20.22 cm) after 2 weeks treatment and of 5.4% after 4 weeks.

### Instrumental Evaluations

The hydration and the principal torsiometric parameters—Ue (immediate extensibility), Uf (final extensibility), Uv (viscoelasticity) and Ur (immediate elastic recovery)—were determined at T0, after 30 minutes (T30 min) and after 2 hours (T2 h) from the first application in order to make a short term evaluation, and at T2 (at least 8 hours after the last application of the shock treatment) and at T4 (at least 8 hours after the last application) in order to make a long term evaluation.

### Torsiometry

The results are reported in the following tables:

TABLE 1
Short term evaluation

| | Percentage of | |
|---|---|---|
| | improvement | |
| | vs baseline | |
| | T30min | T2h |
| Ue | 5.9% | 11.5% |
| Uf | 8.1% | 11.7% |
| Uv | 11.8% | 12% |
| Ur | 21.3% | 6.9% |

TABLE 2
Long term evaluation

| | Percentage of | |
|---|---|---|
| | improvement | |
| | vs baseline | |
| | T2 | T4 |
| Ue | −8.6% | −19.1% |
| Uf | −6.2% | −16.2% |
| Uv | −1.9% | −11.3% |
| Ur | −5% | −14.8% |

The short term results show the effectiveness of the shock treatment on skin elasticity; it was indeed observed a significant improvement of all the examined parameters at T30 min and T2 h.

The long term evaluation underlines the significant firming activity of the tested compositions. This activity, already present at T2, resulted indeed still more remarkable at the end of the study.

### Hydration

The results are reported in the following table:

TABLE 3
Percentage of improvement vs baseline

| | T30min | T2h | T2 | T4 |
|---|---|---|---|---|
| Skin hydration | +11.6% | +11.3% | +13.9% | +21.4% |

These results underline the remarkable and significant hydrating properties of the tested compositions. The improvement of skin hydration obtained after the first application was indeed confirmed after 2 or 4 weeks treatment. Moreover, the increase of the hydration level obtained at the end of the study resulted statistically more pronounced in comparison with the obtained results at T2 underlying the strengthening effect of the maintenance treatment.

The anti-age treatment effectiveness on the neck and décolleté was shown with a study conducted on 25 female healthy subjects, age range 45-68 (mean=58) with a light-moderate cutaneous ageing on the neck and décolleté.

The experiment foresaw three visits: a basal visit (T0), a visit after 2 weeks treatment (T2) and a final visit after 4 weeks treatment (T4). Moreover, a short term evaluation was performed (only for torsiometry and hydration) 30 minutes and 2 hours after the first application of the product.

The following evaluations were performed:

### Clinical Evaluations

décolleté: wrinkles, skin brightness and cutaneous microrelief
neck: skin slackness and skin tonicity

### Instrumental Evaluations

Torsiometry, profilometry, face coordinates evaluation-ptosis.

### Clinical Evaluations

Décolleté

After 4 weeks treatment a significant and remarkable decrease of roughness grade (at least 0.5 point) compared with basal conditions was observed in 72% of the subjects.

The cutaneous microrelief looked more regular already after 2 weeks treatment with an improvement at least of one point in 48% of the subjects. This result appeared still more evident at the end of the study with an improvement of basal conditions in 88% of the subjects.

A remarkable increase of décolleté brightness was observed in 48% of the subjects after 2 weeks and in 80% of the subjects after 4 weeks.
Neck

After 2 weeks and at the end of the treatment a significant decrease of the skin slackness was observed in 56% and 88% of the subjects, respectively. The neck appeared more tonic, and in fact after 4 weeks treatment, the pinching resistance, the recovery after pinching and the traction resistance were significantly improved in 56% of the subjects as regards the first two parameters and in 68% of the subjects as regards the third parameter.

### Instrumental Evaluations

### Skin Profilometry

The image analysis of décolleté wrinkles at T0, T2 and T4 showed:
a remarkable reduction of irregularity (Ra) from the average basal value of 9.44 to the value of 6.90 at T2 and of 5.20 at T4 corresponding to 27% reduction at T2 and to 44.9% reduction at T4.
a remarkable reduction of the maximum depth of wrinkles (Rt) from the basal value of 50.80 to the value of 35.90 at T2 and of 29.66 at T4 corresponding to 29.3% reduction at T2 and to 41.6% reduction at T4.

The reduction of these parameters proved not only the efficacy in reducing the number of fine wrinkles but also their decreased depth.
a remarkable increase of the average depth of wrinkles (Rz) from the basal value of 195.07 to the value of 202.42 at T2 and of 208.23 at T4 corresponding to an increase of 3.8% at T2 and of 6.7% at T4. This value was calculated on the total number of wrinkles, consequently the reduction of fine wrinkles results in an increase of Rz.
a remarkable increase of the minimum height of wrinkles (Rmin) from the basal value of 164.41 to the value of 180.97 at T2 and of 191.24 at T4 corresponding to an increase of 10% at T2 and of 16.3% at T4. This shows that the wrinkles under the medium value disappeared at the end of the treatment.

### Face Coordinates (Ptosis)

The analysis of face coordinates at T0, T2 and T4 has shown a significant variation confirming the “lifting” efficacy of the treatment.

### Torsiometry and Hydration

The hydration and the main torsiometric parameters—Ue (immediate extensibility), Uf (final extensibility), Uv (viscoelasticity) and Ur (immediate elastic recovery)—were performed at T0, after 30 minutes (T30) and after 2 hours (T2 h) from the first application for a short term evaluation and at T2 and T4 for a long term evaluation at the décolleté level.

### Torsiometry

The results are reported in the following tables:

TABLE 4
Short term evaluation

| | Percentage of | |
|---|---|---|
| | improvement | |
| | vs baseline | |
| | T30min | T2h |
| Ue | −5% | −16% |
| Uf | −4.7% | −19.4% |
| Uv | −4.3% | −24% |
| Ur | −5.8% | −10% |

TABLE 5
Long term evaluation

| | Percentage of | |
|---|---|---|
| | improvement | |
| | vs baseline | |
| | T2 | T4 |
| Ue | 1.8% | −12.2% |
| Uf | −4.3% | −20.6% |
| Uv | −12.8% | −32.3% |
| Ur | −5.5% | −3.1% |

The results showed the efficacy of the tested compositions on skin elasticity; indeed the décolleté skin appeared smoother and more stretched.

1. (canceled)

2. A method for re-shaping treatment of a breast, comprising applying a cosmetic formulation containing proanthocyanidin A2 and its complexes with phospholipids on the breast.

3. A method for the anti-age treatment of the skin of the neck and the décolleté, comprising applying a cosmetic formulation containing proanthocyanidin A2 and its complexes with phospholipids to the skin of the neck and the décolleté.

4-6. (canceled)

7. Method according to claim 2, wherein the proanthocyanidin A2 is complexed with phospholipids.

8. Method according to claim 2, wherein the proanthocyanidin A2 is complexed with phosphatidylcholine.

9. Method according to claim 3, wherein the proanthocyanidin A2 is complexed with phospholipids.

10. Method according to claim 3, wherein the proanthocyanidin A2 is complexed with phosphatidylcholine.

11. Method according to claim 3, wherein said cosmetic composition contains an amount of proanthocyanidin A2 or of a complex thereof ranging from 0.1% to 5% w/w.

12. Method according to claim 11, wherein said cosmetic composition contains an amount of proanthocyanidin A2 or of a complex thereof ranging from 0.5% to 3% w/w.

13. Method according to claim 12, wherein said cosmetic composition contains an amount of proanthocyanidin A2 or of a complex thereof ranging from 2% to 2.5% w/w.

14. Method according to claim 2, wherein said cosmetic composition contains an amount of proanthocyanidin A2 or of a complex thereof ranging from 0.1% to 5% w/w.

15. Method according to claim 14, wherein said cosmetic composition contains an amount of proanthocyanidin A2 or of a complex thereof ranging from 0.5% to 3% w/w.

16. Method according to claim 15, wherein said cosmetic composition contains an amount of proanthocyanidin A2 or of a complex thereof ranging from 2% to 2.5% w/w.
